(19) United States
(12) Patent Application Publication
(10) Pub. No.: US 2010/0280594 A1
(43) Pub. Date: Nov. 4, 2010

(54) Antithrombotic Neurovascular Device Containing a Glycoprotein IIB/IIIA Receptor Inhibitor for The Treatment of Brain Aneurysms and/or Acute Ischemic Stroke, and Methods Related Thereto

(75) Inventors: Ronald A. Sahatjian, Lexington, MA (US); Ajay K. Wakhloo, Wellesley, MA (US)

Correspondence Address: LUCY ELANDJIAN;IP LAW SERVICES, LLC., 450 E. Waterside Drive, Suite 202, Chicago, IL, 60601, US

(73) Assignee: MEDI-SOLVE, LLC, Lexington, MA (US)

(21) Appl. No.: 12/772,000

(22) Filed: Apr. 30, 2010

Related U.S. Application Data
(60) Provisional application No. 61/215,025, filed on May 1, 2009.

(51) Int. Cl. A61F 2/82; B05D 7/00

(52) U.S. Cl. 623/1.18; 623/1.46; 427/2.25

(57) ABSTRACT

Disclosed is an antithrombotic neurovascular device useful for the treatment of brain aneurysms and/or acute ischemic stroke. The device comprises a mechanical structure, which may be a stent, stent-like structure, or flow diverter, and a drug-eluting coating. This device is designed for local delivery of antiplatelet medication to the site of device implantation in order to improve outcomes for the population of brain aneurysm and/or acute ischemic stroke patients currently being treated with stents, and for use with patients presenting with ruptured aneurysms, in whom systemic dual antiplatelet therapy is contraindicated. The coating comprises an antiplatelet drug, preferably, a GPIIb/IIIa receptor inhibitor, more preferably, abcixmab, and optionally comprises a polymeric binder that functions as a drug modulating polymer. Also disclosed are methods for producing the antithrombotic neurovascular device, and using the device for the treatment of brain aneurysms and/or acute ischemic stroke.

### CROSS REFERENCE TO RELATED APPLICATION

This is a non-provisional application claiming the benefit of and priority to provisional patent application Ser. No. 61/215,025, filed on May 1, 2009, which is incorporated herein by reference.

### TECHNICAL FIELD

The present invention relates generally to antithrombotic neurovascular devices, and methods related thereto. Specifically, the present invention pertains to an antithrombotic neurovascular device, containing a glycoprotein IIb/IIIa receptor inhibitor, for the treatment of brain aneurysm and/or acute ischemic stroke. The device of the present invention serves to reduce stent thrombosis and stent-associated thromboembolism, and reduce bleeding risk as compared to systemic dual-antiplatelet therapy.

### BACKGROUND

Aneurysmal subarachnoid hemorrhage (SAH) is a devastating event that affects approximately 30,000 Americans each year. Although less common than ischemic stroke, the case fatality rate for subarachnoid hemorrhage is substantially higher (35-50 percent vs. 8-12 percent), and as SAH occurs more frequently than ischemic stroke among younger patients, the number of years of potential life lost before age 65 is similar (i.e., 2-5 years). Of those patients who survive, 40 percent will do so with significant long-term disability.

Endovascular brain aneurysm obliteration is a rapidly evolving, minimally invasive procedure for the treatment and prevention of SAH. A wide variety of medical devices are now available for the treatment of intracranial aneurysms. The standard surgical approach entails after craniotomy, the placement of a clip across the neck of an aneurysm to exclude it from the main circulation. The goal is to prevent a (re)bleed into the brain from an aneurysm rupture. The standard surgical approach is being replaced by minimally endovascular techniques. Most of such techniques involve the placement of detachable platinum coils within the aneurysm pouch, which detach either mechanically or via the use of electrically detachable systems. This intravascular approach to aneurysm treatment is utilized to create, after coiling the sac, a stable thrombus and subsequent scarring within the aneurysm pouch. This prevents rupture which can be fatal. The placement of coils however, has shown to have some intrinsic limitations, one of the most important being the risk of aneurysm re-canalization within the first year due to coil compaction or to large size of aneurysm neck. The second most common risk is rupture of the aneurysm during placement of coils within the extremely thin-walled aneurysm sac. In general, this leads to severe neurological deficit or even the death of a patient. Multiple flow studies over the past decade have shown that redirecting blood flow within the parent artery away from the aneurysm may lead to a safe and permanent thromboocclusion of the aneurysm without the risk of damaging the aneurysm wall.

Tubular structures or stents have been developed to address these issues. However, most of the current devices following that concept (Neuroform®, product of Boston Scientific Corp., Natick, Mass.; and, Enterprise®, product of Cordis-Johnson & Johnson Corp., East Bridgewater, N.J.) work only in conjunction with coils because of their high porosity (ratio of material free area to area covered by material). Stent placement is necessary in many cases as an adjunct to neurovascular coil placement, and clinical evidence that demonstrates substantially improved aneurysm treatment due to the hemodynamic effects of stents and other stent-like neurovascular devices is amassing.

However, there is a significant need to improve the safety of aneurysm treatment with these devices. Stent thrombosis and stent-associated thromboembolism are the most common causes of complications during and after stent placement. Preprocedural and long-term systemic dual antiplatelet therapy is the current standard of care for the prevention of these events, but there are significant limitations to the safety and efficacy of this approach. At standard antiplatelet doses, clinically evident thromboembolic events occur in as many as 10 percent of the cases, and “silent” thromboembolic events, the true significance of which is unknown, occur in as many as 25 percent of the cases. The systemic inhibition of platelet function predisposes patients to hemorrhagic complications, many of which occur in vascular territories outside of the therapeutic target. Platelet adhesion and aggregation is believed to be an important part of thrombus formation. This activity is mediated by a number of platelet adhesive glycoproteins. The binding sites for fibrinogen, fibronectin and other clotting factors have been located on the platelet membrane glycoprotein complex IIb/IIIa (GPIIb/IIIa). When a platelet is activated by an agonist, such as thrombin, the GPIIb/IIIa receptor site becomes available to fibrinogen, and eventually resulting in platelet aggregation and clot formation.

One approach to blocking these thrombus formation sites has been via the use of various therapeutic agents administered systemically. However, increased risk of hemorrhage associated with such therapy limits the use of these medications in patients presenting with aneurysms that have already ruptured, which in turn limits the treatment of such patients using stents or stent-like devices.

### SUMMARY OF THE INVENTION

While systemic dual antiplatelet therapy is the current standard of care for patients undergoing coiling of brain aneurysms and/or acute ischemic stroke, there is a risk of complications associated with such therapy, including hemorrhagic stroke.

In view of the above, there is a need for an antithrombotic neurovascular device, such as a stent, containing a coating that releases an anti-platelet medication and prevents thromboembolic complications while reducing and potentially eliminating the risk of hemorrhagic complications associated with systemic dual antiplatelet therapy. There is also a need for a method of producing such an antithrombotic neurovascular device.

It is, therefore, an aspect of the present invention to provide a drug-eluting neurovascular device that contains a GPIIb/IIIa receptor inhibitor for the population of aneurysm patients currently being treated with stents and to expand the use of this important technology to patients presenting with the risk of ruptured aneurysms in whom systemic dual antiplatelet therapy is contraindicated.

It is another aspect of the present invention to provide a neurovascular device that comprises a polymer coating that elutes an antiplatelet medication in order to prevent thrombosis and thromboembolism.

It is another aspect of the present invention to provide a drug-eluting neurovascular stent or stent-like structure that delivers antiplatelet medication directly to its implantation site.

It is another aspect of the present invention to provide a drug-eluting flow diverter for neurovascular aneurysm treatment.

It is also an aspect of the present invention to provide a method for producing a neurovascular device comprising a drug-eluting coating such that the coating facilitates controlled release of a GPIIb/IIIa receptor inhibitor.

It is a further aspect of the present invention to provide a method for treating brain aneurysms and/or acute ischemic stroke.

The present invention pertains to an antithrombotic neurovascular device, such as a stent, stent-like structure, and flow diverter, for local delivery of antiplatelet medication to the site of device implantation. The neurovascular device comprises a drug-eluting coating to facilitate reduction of stent thrombosis and stent-associated thromboembolism, and to reduce the risks for bleeding as compared to systemic dual-antiplatelet therapy. The coating is applied to the neurovascular device such that the GPIIb/IIIa receptor inhibitor is released from the device and into the tissue at the site of implantation, preferably, released in a controlled manner. The present device improves outcomes for the population of brain aneurysm patients currently being treated with stents, and expands the use of this important technology to patients presenting with ruptured aneurysms, in whom systemic dual antiplatelet therapy is contraindicated.

In one embodiment of the present invention, the neurovascular device is a stent coated with a GPIIb/IIIa receptor inhibitor that releases into the tissue to inhibit thrombus formation.

In one embodiment of the present invention, the neurovascular device is a stent-like structure comprising a coating capable of releasing a GPIIb/IIIa receptor inhibitor into the tissue, at the site of stent implantation, to inhibit thrombus formation.

In one embodiment of the present invention, the neurovascular device is a flow diverter capable of preventing thrombosis and thromboembolism by employing a polymer coating that elutes an antiplatelet medication.

The present invention also pertains to a method for coating the mechanical structure of the neurovascular device, preferably, to allow for controlled release of GPIIb/IIIa receptor inhibitors. The controlled release of the GPIIb/IIIa receptor inhibitors is advantageous for the population of aneurysm patients currently being treated with stents and patients presenting with the risk of ruptured aneurysms in whom systemic dual antiplatelet therapy is contraindicated.

The present invention also pertains to a method for the treatment treating brain aneurysms and/or acute ischemic stroke using the antithrombotic neurovascular device disclosed herein.

The device and methods of the present invention represent a new approach for protecting patients from the thromboembolic complications associated with stent-assisted coil embolization of treating brain aneurysms and/or acute ischemic stroke, and lay the groundwork for continued improvement with future generations of neurovascular devices for neurovascular parent artery repair.

The above summary of the present invention is not intended to describe each illustrated embodiment or every implementation of the present invention. The figures and the detailed description that follow particularly exemplify these embodiments.

### BRIEF DESCRIPTION OF THE DRAWING

The invention may be more completely understood in consideration of the following detailed description of various embodiments of the invention in connection with the accompanying drawing, in which:

FIG. 1 graphically illustrates the rate of elution of the drug from the coating of the neurovascular device for the two coated devices, according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention pertains to an antithrombotic neurovascular device designed to prevent thromboembolic complications while reducing and potentially eliminating the risk of hemorrhagic complications associated with systemic dual antiplatelet therapy. The present invention also pertains to methods for producing the neurovascular device and using the neurovascular device to treat brain aneurysms and/or acute ischemic stroke. The present invention presents a novel approach to protecting patients from the thromboembolic complications associated with stent-assisted coil embolization of brain aneurysms and/or acute ischemic stroke.

### 1. DEFINITIONS

The term “stent-like structure”, as used herein, refers to a tubular mesh structure that bridges across the neck of the aneurysm.

The term “drug” or “antiplatelet medication”, as used herein, refers to an inhibitor of platelet adhesion or a GPIIb/IIIa receptor inhibitor. Drug is preferably a pharmaceutical preparation comprising at least one active agent, and it may also include non-active components.

The term “drug modulating”, as used herein, refers to a means for controlling the release of a drug over a specific period of time.

It is to be understood that the singular forms of “a”, “an”, and “the”, as used herein and in the appended claims, include plural reference unless the context clearly dictates otherwise.

### 2. THE NEUROVASCULAR DEVICE

The present invention pertains to an antithrombotic neurovascular device useful for the treatment of brain aneurysms and/or acute ischemic stroke. The present antithrombotic neurovascular device is designed to improve outcomes for the population of aneurysm patients currently being treated with stents, and to patients presenting with ruptured aneurysms, in whom systemic dual antiplatelet therapy is contraindicated.

The antithrombotic neurovascular device of the present invention comprises a mechanical structure and a coating composition, which is preferably coated onto the surface of the mechanical structure. The mechanical structure of the present device may be a stent, a stent-like structure, or a flow diverter. The mechanical structure may be self-expanding or balloon-expandable. In preferred embodiments, the mechanical structure comprises a configuration capable of self-expansion against the neck of an aneurysm without requiring the use of a balloon. The mechanical structure serves as a scaffold for embolic coils to prevent herniation of the coils into the parent vessel. The device of the present invention is typically used with a delivery system to deliver it to the treatment site in the neurovascular anatomy. Because most aneurysms are located in very distal sections of the inter-crainial anatomy, with very small vessels such a configuration enables its deployment to desired anatomy through the inner diameter of a very small catheter; whereas, a balloon expandable stent would not lend itself for such deployment and thus would not be able to access the desired anatomy.

The mechanical structure may be fabricated from any material suitable for medical applications, including metals, polymers, or any combination thereof. Preferably, the mechanical structure is fabricated from a material comprising a suitable metal, such as stainless steel, inconel, nitinol, cobalt-chromium, or titanium. More preferably, the mechanical structure comprises materials having shape-memory characteristic, such as nitinol. Use of shape-memory materials facilitates the application of self-expanding structures (e.g., self-expanding stents) to applications for which balloon stents would be unsuitable, such as in the treatment of brain aneurysms and/or acute ischemic stroke.

The coating composition of the present device comprises an inhibitor of platelet adhesion drug, particularly, a class of agents referred to as GPIIb/IIIa receptor inhibitors, which are optimal for use herein as they provide a combination of potent antiplatelet activity with favorable pharmacological properties. The GPIIb/IIIa receptor inhibitors preferable for use herein include abcixmab, clopidogrel and eptifibatide, with abcixmab being the preferred GPIIb/IIIa receptor inhibitor. Abcixmab is commercially available as ReoPro® (product of Eli Lilly and Company, Indianapolis, Ind.), clopidogrel is commercially available as Plavix® (product of Sanofi-Aventis Corp., Paris, France), and eptifibatide is commercially available as Integrilin® (product of Millennium Pharmaceuticals, Cambridge, Mass.).

The coating composition may also comprise a binder, preferably, a polymeric binder, to modulate drug release. The polymeric binder may be a biodurable polymer, such as polyurethane, polyethylene vinyl acetate, poly styrene isobutylene styrene, or poly butylmethacrylate, or a bioerodeable polymer, such as a poly lactic acid (PLA), poly glycolic acid (PGA), or a co-polymer thereof, such as poly lactic glycolic acid (PLGA). In coating compositions containing the drug and the binder, the ratio of drug to binder is preferably in the range of from about one to one (1:1) to about one to seven (1:7).

In coatings that comprise a drug and a drug modulating polymer, the drug and the drug modulating polymer may be applied together in a single composition to at least one surface of the mechanical structure, as a single layer, or multiple layers thereof; alternatively, the drug and the drug modulating polymer may be applied as a dual composition system, whereby at least one layer of the composition containing the drug is first coated onto at least one surface of the mechanical structure, and the drug coated surface(s) is then coated with at least one layer of the composition containing the drug modulating polymer.

The neurovascular stent of the present invention differs from cardiovascular stents, for example, cardiovascular stents are used to treat arthrosclerotic disease and do so by propping open a diseased vessel, i.e., functioning as a mechanical structure to keep a blood vessel open. In order to keep a blood vessel open, the cardiovascular stent must open to a desired diameter and have sufficient compression resistance, so as not to collapse from forces in the vessel wall that are trying to re-model the vessel. Further, cardiovascular stents are typically balloon expandable and are delivered to the desired anatomy via a balloon catheter, as self-expanding cardiovascular stents have proven in clinical applications to yield adverse results in coronary vessels. However, the present neurovascular stent need not have such compression resistance, as it is designed and sized to be positioned across the neck of the aneurysm in a patient, where it is used as an adjunct to embolic coil placement within the aneurysm. In wide neck aneurysms, the present device serves as a scaffold for embolic coils to prevent herniation of the coils into the parent vessel. Although the present device, e.g., stent, may comprise any configuration and any material suitable for the purposes disclosed herein, the device is preferably self-expanding, more preferably a metal (e.g., nitinol) mesh, and most preferably in the shape of a tube.

In one embodiment of the present invention, the antithrombotic neurovascular device comprises a self-expanding stent that comprises a coating on its surface, the coating comprising abcixmab. In another embodiment, the coating comprises clopidogrel. In another embodiment, the coating further comprises a polymeric binder, to modulate the release of the drug. In yet another embodiment, the coating comprises abcixmab and an aliphatic (polyester) polyurethane.

In one embodiment of the present invention, the antithrombotic neurovascular device comprises a flow diverter, which is considered a next-generation technology for neurovascular aneurysm treatment. The flow diverter comprises a porous tubular member having a central portion and two ends. The central portion of the tubular member has a sufficiently decreased porosity to block blood flow from entering through the aneurysm, while the two ends have sufficient porosity to keep open small perforator arteries proximate to the intracranial aneurysm. In one embodiment, the flow diverter is constructed according to the disclosure of US 2007/0060994 A1. The flow diverter comprises a coating comprising abcixmab. In another embodiment, the coating further comprises a polymeric binder, to control the release of the drug into the tissue, to prevent clotting of the flow diverter.

The device of the present invention is designed to facilitate the absorption of the drug into the tissue locally, whereby the drug inhibits the formation of platelets. As such, the present device is optimally useful as an adjunct to coiling of cerebral aneurysms.

### 3. METHOD FOR PRODUCING THE NEUROVASCULAR DEVICE

The present invention also pertains to a method for producing the antithrombotic neurovascular device disclosed herein. The present method comprises cleaning the surfaces of the mechanical structure. The cleaning may be accomplished via the use of a medical grade cleaning solution suitable for use herein. The cleaned mechanical structure is coated with a coating composition that comprises an antiplatelet medication, preferably, a GPIIb/IIIa receptor inhibitor, more preferably, abcixmab. The coating composition may also comprise a binder to function as a drug modulating polymer. The coatings in which the composition comprises both a drug and a binder, the composition comprises a mixture of a solution or dispersion or emulsion containing the antiplatelet medication and a solution or dispersion or emulsion containing the drug modulating polymer. The drug modulating polymer is, preferably, a polyurethane, polyethylene vinyl acetate, poly styrene isobutylene styrene, poly butylmethacrylate, poly lactic acid, poly glycolic acid, or poly lactic glycolic acid. The ratio of drug modulating polymer to drug may be selected or modified to control the rate of drug elution from the coating.

The coating composition may be applied as a single composition system (i.e. comprising the drug solution or dispersion or emulsion, or a mixture of the drug and drug modulating polymer solutions or dispersions or emulsions) to at least one surface of the mechanical structure via any suitable coating process, including the use of a coating apparatus, preferably, an apparatus having the capability for controlled rate of application. Preferably, the composition is applied to yield a very precise coating coverage.

The mechanical structure of the present device may also be coated with a dual composition system, such that least one surface of the mechanical structure is first coated with one or more layers of the solution or dispersion or emulsion containing the drug, and then coated surface(s) is coated with one or more layers of the solution or dispersion or emulsion containing the drug modulating polymer.

In one embodiment of the present invention, the coated mechanical structure is a self-expanding stent, preferably, the stent being fabricated from a metallic shape-memory material, and having an expanded diameter in the range of from about 0.5 millimeters (mm) to about 4 mm, preferably, in the range of from about 0.5 mm to about 3 mm.

Having generally described the invention, a more complete understanding thereof may be obtained by reference to the following example that is provided for purposes of illustration only and do not limit the invention.

### Example

Six balloon expandable stents having a diameter of 3 mm (product of Essen Technology Company, Ltd., Beijing, China) were cleaned, using a medical grade soap and water, in preparation to be coated. A five percent (%) solution of Sancure® 1073c, an aliphatic (polyester) polyurethane dispersion (product of Lubrizol Advanced Materials, Inc., Cleveland, Ohio), was prepared by diluting the Sancure® 1073c solution with water. A standard solution of pharmaceutical grade ReoPro®, in single use vials, was procured. ReoPro® is a clear, colorless, sterile, non-pyrogenic solution for intravenous use. The active ingredient in ReoPro® is abciximab, which is a GPIIb/IIIa receptor inhibitor. Each single use vial contains 2 mg/mL of abciximab in a buffered solution (pH 7.2) of 0.01 molar sodium phosphate, 0.15 molar sodium chloride and 0.001% polysorbate 80 (a preservative) in water.

The Sancure® and ReoPro® solutions were added together to yield two separate coating solutions, solution B-1, having a drug to polymer ratio of 50 to 50 (50/50) or one to one (1:1), and solution A-3, having a drug to polymer ratio of 12.5 to 87.5 (12.5/87.5) or one to seven (1:7).

The cleaned stents were coated using a stent coating apparatus, referred to as MediCoat™ system (product of Sono-Tek Corp., Milton, N.Y.), which was utilized in conjunction with the AccuFlow liquid delivery system (product of Sono-Tek Corp.) to provide highly accurate liquid delivery for very precise stent coating coverage. The computer system on the AccuFlow pump was set to deliver 980 microgram (μg) of coating composition from solution B-1 and 2,146 μg of coating composition from solution A-3. The coated stents were air dried in a closed environment at ambient temperature over night.

In order to simulate the release of the drug in tissue, the stents were soaked, to allow the release of the drug, in a phosphate buffered saline solution at PH 7.4 (PBS buffer solution), and aliquots were taken at intervals of 1, 4, 24, and 96 hours. The percentage of drug eluted from each coated stent was calculated based on the amount of abciximab in the aliquot as analyzed by High Performance Liquid Chromatography (HPLC).

The quantity of drug in each coating solution is presented in Table 1, and the quantity of drug eluted from each of the two coated stents are presented in Table 2 and Table 3.

TABLE 1
Quantity of Drug in Coating Solutions

| | | | Quantity of Drug |
|---|---|---|---|
| Solution | Coating | | Loaded |
| B-1 | 980 | μg @ 50/50 (drug/polymer) | 490 μg |
| A-3 | 2146 | μg @ 12.5/87.5 (drug/polymer) | 268.25 μg |

TABLE 2
Quantity of Drug Eluted from Stent Coating (Solution B-1)

| | Quantity of Drug Eluted vs. Time | | | |
|---|---|---|---|---|
| | 1 hour | 4 hours | 24 hours | 96 hours |
| Quantity of | 149.343258 μg | 4.33858 μg | 0.146597 μg | 0.03701 μg |
| Drug Eluted | | | | |

TABLE 3
Quantity of Drug Eluted from Stent Coating (Solution A-3)

| | Quantity of Drug Eluted vs. Time | | | |
|---|---|---|---|---|
| | 1 hour | 4 hours | 24 hours | 96 hours |
| Quantity of | 95.6545826 μg | 3.841238 μg | 0.285441 μg | 0.135511 μg |
| Drug Eluted | | | | |

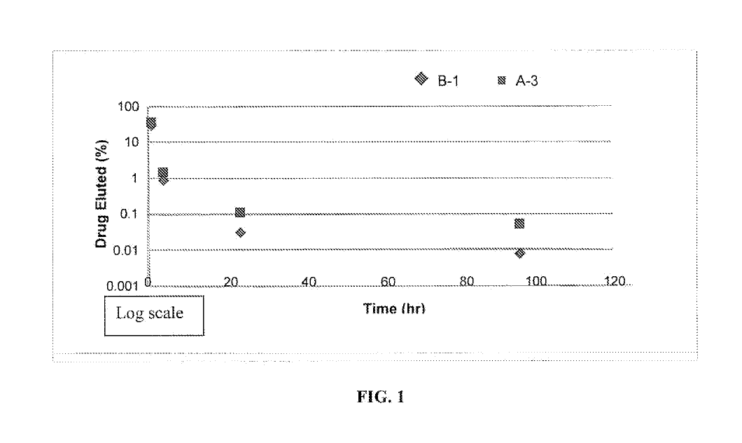

The data in Table 2 and Table 3, as well as the graph in FIG. 1, show two different rates of release of the abciximab from the polyurethane binder, according to the ratio of the drug to the polymeric binder. This data conveys that the abciximab is undergoing a polymer modulated release from the surface of the stent. The release rate can be controlled by adjusting the drug to polymer ratio, depending on the type of aneurysm being treated, the tendency of the patient to clot, as well as other variables.

### 4. METHOD FOR TREATMENT OF BRAIN ANEURYSMS AND/OR ACUTE ISCHEMIC STROKE

The present invention also pertains to a method for treating brain aneurysms and/or acute ischemic stroke. The treatment method involves local delivery of antiplatelet medication to the site of device implantation, which serves to reduce thrombosis and thromboembolism (e.g., stent thrombosis and stent-associated thromboembolism), as well as to reduce the risks for bleeding, as compared to systemic dual-antiplatelet therapy. Patients that have a brain aneurysm and/or an acute ischemic stroke are typically at risk of excessive bleeding, and thus cannot take blood-thinning (antiplatelet and/or anticoagulation) drugs to help prevent stent thrombosis and/or stent-associated thromboembolism. For such patients, local delivery of anti-platelet medications would be the preferred means of therapy. Such therapy will require only a small dosage of drug that does not place the patient at risk for bleeding from hemorrhage associated aneurysms or bleeding in vascular territories outside of the therapeutic target.

The treatment method comprises implanting in a patient the antithrombotic neurovascular device disclosed herein across the neck of an aneurysm, in order to stabilize the device. A delivery system, typically composed of an introducer and delivery wire, may be utilized to traverse the neurovascular anatomy in the placement of the device. A set of embolic coils may be placed in the aneurysm to prevent further blood flow into the aneurysm by creating a stable thrombus and subsequent scarring, which prevents rupture of the aneurysm. The implanted device releases the antiplatelet medication into the tissue at the site of implantation. Preferably, the device coating comprises a drug modulating polymer to facilitate controlled release of the drug.

In one embodiment of the present invention, a neurovascular self-expanding stent comprising a drug-eluting coating containing a GPIIb/IIIa receptor inhibitor is used to treat a brain aneurysm. The stent is used in conjunction with a stent delivery system. The stent is thus implanted in a patient across the neck of an aneurysm according to the FDA approved method for coiling of aneurysms. The stent releases the GP IIb/IIIa receptor inhibitor into the tissue at the site of implantation in a controlled manner. A set of embolic coils are also placed in the aneurysm. In another embodiment, the GPIIb/IIIa receptor inhibitor is abciximab. In yet another embodiment, the GPIIb/IIIa receptor inhibitor is clopidogrel.

In one embodiment, a neurovascular flow diverter comprising a drug-eluting coating, containing a GPIIb/IIIa receptor inhibitor and a drug modulating polymer, is implanted in a patient across the neck of the aneurysm. Due to the central portion of the flow diverter being a tubular member having a sufficiently decreased porosity, it can block blood flow from entering through the aneurysm. The drug modulating polymer controls the release of the GPIIb/IIIa receptor inhibitor into the tissue at the site of implantation, and serves to prevent clotting of the flow diverter. The GPIIb/IIIa receptor inhibitor is abciximab, and the drug modulating polymer is a polyurethane.

As noted above, the present invention pertains to an antithrombotic neurovascular device, containing a drug-eluting coating comprising a glycoprotein IIb/IIIa receptor inhibitor, for the treatment of brain aneurysm and/or acute ischemic stroke, and methods related thereto. The present invention should not be considered limited to the particular embodiments described above, but rather should be understood to cover all aspects of the invention as fairly set out in the appended claims. Various modifications, equivalent processes, as well as numerous structures to which the present invention may be applicable will be readily apparent to those skilled in the art to which the present invention is directed upon review of the present specification. The claims are intended to cover such modifications.

We claim:

1. An antithrombotic neurovascular device for the treatment of brain aneurysm and/or acute ischemic stroke, said device comprising a mechanical structure and a coating composition coated onto at least one surface of the mechanical structure, said coating composition comprising a GPIIb/IIIa receptor inhibitor, said mechanical structure being a stent, a stent-like structure, or a flow diverter.

2. An antithrombotic neurovascular device according to claim 1, said coating composition comprising a drug modulating polymer.

3. An antithrombotic neurovascular device according to claim 1, said GPIIb/IIIa receptor inhibitor comprising abciximab.

4. An antithrombotic neurovascular device according to claim 1, said GPIIb/IIIa receptor inhibitor comprising clopidogrel.

5. An antithrombotic neurovascular device according to claim 2, said drug modulating polymer comprising a polyurethane, polyethylene vinyl acetate, poly styrene isobutylene styrene, poly butylmethacrylate, poly lactic acid, poly glycolic acid, or poly lactic glycolic acid.

6. An antithrombotic neurovascular device according to claim 2, said drug modulating polymer comprising aliphatic (polyester) polyurethane.

7. An antithrombotic neurovascular device according to claim 1, said device being a stent or stent-like structure comprising material having shape-memory characteristics.

8. An antithrombotic neurovascular device according to claim 7, said device being a self-expanding stent having an expanded diameter in the range of from about 0.5 mm to about 4 mm.

9. An antithrombotic neurovascular device according to claim 1, said device being a self-expanding stent comprising a coating and having an expanded diameter in the range of from about 0.5 mm to about 4 mm, said coating comprising abciximab and a drug modulating polymer.

10. A method for producing an antithrombotic neurovascular device comprising coating at least one surface of a mechanical structure with a coating composition that comprises a GPIIb/IIIa receptor inhibitor, said mechanical structure being a stent, a stent-like structure, or a flow diverter.

11. A method according to claim 10, said coating composition comprising a drug modulating polymer.

12. A method according to claim 10, comprising coating the at least one surface coated with the composition that comprises a GPIIb/IIIa receptor inhibitor with a composition comprising a drug modulating polymer.

13. A method according to claim 10, said mechanical structure being a self-expanding stent having an expanded diameter in the range of from about 0.5 mm to about 4 mm.

14. A method according to claim 10, said GPIIb/IIIa receptor inhibitor comprising abciximab.

15. A method according to claim 10, said GPIIb/IIIa receptor inhibitor comprising clopidogrel.

16. A method according to claim 11, said drug modulating polymer comprising a polyurethane, polyethylene vinyl acetate, poly styrene isobutylene styrene, poly butylmethacrylate, poly lactic acid, poly glycolic acid, or poly lactic glycolic acid.

17. A method according to claim 11, said drug modulating polymer comprising aliphatic (polyester) polyurethane.

18. A method for the treatment of brain aneurysm and/or acute ischemic stroke, comprising implanting in a patient an antithrombotic neurovascular device according to claim 1 across the neck of an aneurysm.

19. A method for the treatment of brain aneurysm and/or acute ischemic stroke, comprising implanting in a patient an antithrombotic neurovascular device according to claim 2 across the neck of an aneurysm.

20. A method according to claim 18, comprising placing a set of embolic coils in the aneurysm.
